(19) United States
(12) Patent Application Publication
(10) Pub. No.: US 2018/0127159 A1
(43) Pub. Date: May 10, 2018

(54) BEVERAGE CONTAINER HAVING FRAGRANT COMPONENT

(71) Applicant: Randy Mark Cunningham, Roseville, CA (US)

(72) Inventor: Randy Mark Cunningham, Roseville, CA (US)

(21) Appl. No.: 15/600,674

(22) Filed: May 19, 2017

Related U.S. Application Data
(60) Provisional application No. 62/476,443, filed on Mar. 24, 2017.
(60) Provisional application No. 62/417,673, filed on Nov. 4, 2016.

(51) Int. Cl. B65D 41/34; B65D 51/16; A61L 9/01; A23L 2/56

(52) U.S. Cl. CPC B65D 41/3428; B65D 51/1688; B65D 41/32; A23L 2/56; A61L 9/01

(57) ABSTRACT

A beverage container having a fragrant component, and related materials and methods of manufacturing the beverage container, are described. The beverage container may be a bottle assembly including the fragrant component between a finish of the bottle and a cap. The fragrant component may be encased and protected between the cap and the finish, and upon removal of the cap, the fragrant component may remain on the finish and be exposed to release a fragrance. The fragrance can contribute to an experience of a consumer when the consumer drinks a beverage from the bottle. Other embodiments are also described and claimed.

This application claims the benefit of priority of U.S. Provisional Patent Application No. 62/476,443, filed Mar. 24, 2017, and U.S. Provisional Patent Application No. 62/417,673, filed Nov. 4, 2016, and incorporates herein by reference those provisional patent applications.

### BACKGROUND

### Field

Embodiments related to beverage packaging are disclosed. More particularly, embodiments related to beverage containers, are disclosed.

### Background Information

Current trends in beverage consumption have influenced a rise in obesity in the world. Researchers have linked trends in obesity and diabetes to an increase in the consumption of sugar-fortified carbonated beverages. To support consumer's efforts to consume healthier beverages, numerous companies have promoted and offered both “low sugar” and “no sugar” alternative beverages. A widely available “no sugar” healthy alternative beverage is bottled water. Bottled water is typically tasteless, however, flavorings can be added to give the bottled water a desired taste. Enhancing the taste of water can spur consumers to choose healthier bottled water beverages over less-healthy options.

### SUMMARY

Embodiments related to beverage container assemblies having a fragrant component between a container and a cover are described. In an embodiment, a bottle assembly includes a bottle, a cap, and a fragrant component between the cap and the bottle. For example, the fragrant component may be mounted on the bottle between the cap and a finish of the bottle. The fragrant component can include a fragrant material substance that emits a fragrance when the cap is removed from the bottle. In an embodiment, the fragrant material system is a mixture of a base substance and a fragrant substance, and the mixture may be non-flowable at room temperature. Accordingly, the fragrant material system may be loaded into holes in a sleeve of the fragrant component, and remain in the holes during distribution and use of the bottle assembly. When a user drinks a beverage from the bottle, a taste of the beverage may combine with a smell of the fragrant material system to yield a pleasing synergy of sensations.

The fragrant component may be coupled to the cap or the bottle by one or more interlock features. For example, the sleeve of the fragrant component may include an interlock groove that snaps over a retention bump on the bottle. Alternatively, the sleeve may include an interlock thread that can be screwed onto a mating thread on the bottle. In an embodiment, the fragrant component does not include a sleeve, e.g., the fragrant component may be a mixture of the base substance and fragrant substance that is dispensed directly onto a surface of the cap or bottle and retained on the surface by adhesion. The fragrant component may include a fragrant tape that can be stuck to the bottle.

A method of manufacturing the bottle assembly having a fragrant component may include mounting the fragrant component on one or more of the bottle or the cap, and fastening the cap to the bottle to encase the fragrant component between the cap and the bottle. Mounting the fragrant component can include mounting a sleeve containing the fragrant material system on the finish of the bottle. The bottle may be filled with a beverage before or after mounting the fragrant component.

The above summary does not include an exhaustive list of all aspects of the present invention. It is contemplated that the invention includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

FIG. 1 is a pictorial view of a bottle assembly, in accordance with an embodiment.

FIG. 2A is a cross-sectional view of a bottle subassembly including a fragrant component within a cap, in accordance with an embodiment.

FIG. 2B is a detail view, taken from Detail A of FIG. 2A, of a fragrant material system, in accordance with an embodiment.

FIG. 3 is a side view of a fragrant component having a retention groove, in accordance with an embodiment.

FIG. 4 is a cross-sectional view, taken about line A-A of FIG. 3, of a fragrant component having a retention groove, in accordance with an embodiment.

FIG. 5 is a side view of a bottle finish having a retention bump, in accordance with an embodiment.

FIG. 6 is a partial cross-sectional view of a bottle assembly including a fragrant component having a retention groove between a cap and a bottle, in accordance with an embodiment.

FIG. 7 is a detail view, taken from Detail B of FIG. 6, of a bottle assembly including a fragrant component having a retention groove between a cap and a bottle, in accordance with an embodiment.

FIG. 8 is a side view of a tamper evident band and a fragrant component having a retention groove retained on a bottle finish, in accordance with an embodiment.

FIG. 9 is a cross-sectional view of a bottle subassembly including a fragrant component integrated with a tamper evident band of a cap, in accordance with an embodiment.

FIG. 10 is a side view of a bottle finish having a locking flange, in accordance with an embodiment.

FIG. 11 is a side view of a fragrant component integrated with a tamper evident band retained on a bottle finish, in accordance with an embodiment.

FIG. 12 is a cross-sectional view of a bottle subassembly including a fragrant component within a cap, in accordance with an embodiment.

FIG. 13 is a side view of a fragrant component having an outer retention flange, in accordance with an embodiment.

FIG. 14 is a cross-sectional view, taken about line B-B of FIG. 13, of a fragrant component having an outer retention flange, in accordance with an embodiment.

FIG. 15 is a side view of a bottle finish, in accordance with an embodiment.

FIG. 16 is a cross-sectional view of a bottle assembly including a fragrant component between a cap having a plug and a bottle, in accordance with an embodiment.

FIG. 17 is a detail view, taken from Detail C of FIG. 16, of a bottle assembly including a fragrant component between a cap having a plug and a bottle, in accordance with an embodiment.

FIG. 18 is a side view of a tamper evident band and a fragrant component retained on a bottle finish, in accordance with an embodiment.

FIG. 19 is a cross-sectional view of a bottle subassembly including a fragrant component screwed into a cap, in accordance with an embodiment.

FIG. 20 is a side view of a fragrant component having a thread, in accordance with an embodiment.

FIG. 21 is a cross-sectional view, taken about line C-C of FIG. 20, of a fragrant component having a thread, in accordance with an embodiment.

FIG. 22 is a side view of a bottle finish having a thread, in accordance with an embodiment.

FIG. 23 is a partial cross-sectional view of a bottle assembly including a fragrant component having a thread between a cap and a bottle, in accordance with an embodiment.

FIG. 24 is a side view of tamper evident band and a fragrant component having a thread retained on a bottle finish, in accordance with an embodiment.

FIG. 25A is a side view of a fragrant tape on a bottle finish, in accordance with an embodiment.

FIG. 25B is a cross-sectional view, taken about line D-D of FIG. 25A, of a fragrant tape, in accordance with an embodiment.

FIG. 26 is a partial cross-sectional view of a bottle assembly having a fragrant tape between a cap and a bottle, in accordance with an embodiment.

FIG. 27 is a side view of a fragrant tape between threads of a bottle, in accordance with an embodiment.

FIG. 28 is a partial cross-sectional view of a bottle assembly having a fragrant tape between a cap and a bottle, in accordance with an embodiment.

FIG. 29 is a top view of a fragrant tape on a can, and a removed can cover, in accordance with an embodiment.

FIG. 30 is a side view of a fragrant tape on a sports cap nipple, in accordance with an embodiment.

FIG. 31 is a side view of a fragrant tape on a bottle neck, and a removed bottle cover, in accordance with an embodiment.

FIG. 32 is a side view of a bottle finish having a groove, in accordance with an embodiment.

FIG. 33 is a cross-sectional view, taken about line E-E of FIG. 32, of a bottle finish having a groove, in accordance with an embodiment.

FIG. 34 is a partial cross-sectional view of a bottle assembly having a fragrant thread between a cap and a groove of a bottle finish, in accordance with an embodiment.

FIG. 35 is a side view of a fragrant thread retained in a groove of a bottle finish, in accordance with an embodiment.

FIG. 36 is a perspective view of a fragrant material system retained in a groove on a can, and a removed can cover, in accordance with an embodiment.

FIG. 37 is a side view of a sports cap nipple having a groove, in accordance with an embodiment.

FIG. 38 is a side view of a fragrant ring retained in a groove of a sports cap nipple, in accordance with an embodiment.

FIG. 39 is a flowchart of a method of manufacturing a bottle assembly having a fragrant component, in accordance with an embodiment.

### DETAILED DESCRIPTION

Embodiments describe a beverage container having a fragrant component, and related materials and methods of manufacturing the beverage container. The beverage container may hold water and the fragrant component may release an enjoyable fruit scent, such as citrus or berry fragrances. The beverage container may hold other beverages and the fragrant component may release a different scent, however. For example, the beverage container may hold a tea, such as iced tea, and the fragrant component may release a complementary lemon aroma. Accordingly, the containers, beverages, and fragrances described below are provided by way of example and not limitation.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “above” may indicate a first direction relative to a point of interest. Similarly, “below” may indicate a second direction opposite to the first direction. Such terms are provided to establish relative frames of reference, however, and are not intended to limit the use or orientation of a beverage container to a specific configuration described in the various embodiments below.

Bottled water represents a healthy beverage choice, however, some consumers do not choose to buy bottled water because it is viewed as being more bland than, for example, sugar-fortified carbonated beverages. Attempts at promoting and selling small squeeze bottles with fruit flavored concentrate for consumers to add to bottled water have not overcome this impression for several reasons. First, squeeze bottles of fruit flavored concentrate require additional expenditures by the consumer, and second, it is inconvenient to have to carry a squeeze bottle to use whenever one wants to flavor a bottle of water.

In an aspect, a beverage container including a fragrant component to enhance a tasting experience of a user, is provided. The fragrant component can include a fragrant material system that incorporates fragrant substances, e.g., aromatic compounds or fragrant compounds. The fragrant component can be covered or protected by a cap of the beverage container during distribution of a beverage, and when the user removes the cap, the fragrant component can release a fragrance having a predetermined smell. More particularly, the predetermined smell can complement the taste of the beverage contained in the beverage container such that, when the user drinks the beverage, the user receives an enhanced sensory experience. The cap can be placed onto the beverage container to cover and protect the fragrant component until the user wants another drink. Thus, the beverage container having a fragrant component within a cap: provides a reliably re-sealable closure; provides a tamper evident closure; provides surfaces for printing text or graphic images; and enhances a sensory experience of a user when the consumer drinks a beverage from the beverage container. By enhancing users' drinking experiences, the beverage container and/or fragrant component can make healthy beverage options more appealing to consumers, which can have a positive contribution to the public goal of reducing obesity in the world.

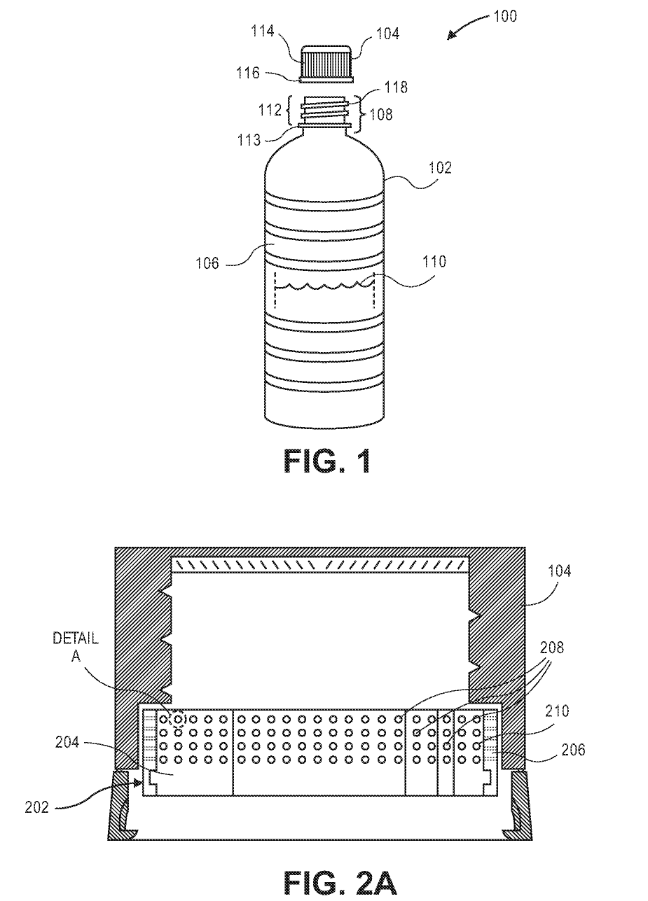

Referring to FIG. 1, a pictorial view of a bottle assembly is shown in accordance with an embodiment. A bottle assembly 100 represents one type of beverage container, and other containers include cans, as described below. Bottle assembly 100 may include a bottle 102, and a cap 104. For example, bottle 102 may be a plastic beverage bottle fabricated by a blow molding process. More particularly, bottle 102 may be molded from a light durable plastic, such as polyethylene terephthalate (PETE). Bottle 102 can include a body 106, and an upper end referred to as a finish 108. In an embodiment, body 106 of bottle 102 is filled with a beverage 110, and a user may drink or dispense the beverage 110 through a mouth 112 of finish 108. A variety of beverages can be contained in and distributed by bottle 102. For example, beverage 110 may include water, ice tea, fruit juices, or carbonated beverages. In addition to having mouth 112, finish 108 may include fasteners, such as a threaded surface or a locking flange 113 to interlock with a portion of cap 104.

Cap 104 may include a screwtop 114 and a tamper evident band 116 fabricated as a single component by an injection molding or compression molding process. The component can be molded from polymer resins, such as polypropylene. After molding, cap 104 can be subjected to a folding process and a peripheral scoring process to create a line of weakness at a junction between screwtop 114 and tamper evident band 116.

Cap 104 can interface with the threaded surface of finish 108, e.g., a mouth thread 118, to seal beverage 110 inside of bottle 102. Tamper evident band 116 can engage locking flange 113 on finish 108 when cap 104 is screwed onto mouth thread 118 on mouth 112. When cap 104 is unscrewed from the thread, screwtop 114 and tamper evident band 116 may separate at the line of weakness, allowing screwtop 114 to be removed from bottle 102 and tamper evident band 116 to be retained on finish 108. The separation process can provide visual or tactile feedback to alert the user when tampering with bottle 102 or beverage 110 has occurred. Accordingly, cap 104 can be mounted on mouth 112, and can be reversibly removed and replaced to open or re-seal bottle 102 whenever the user wants to drink beverage 110.

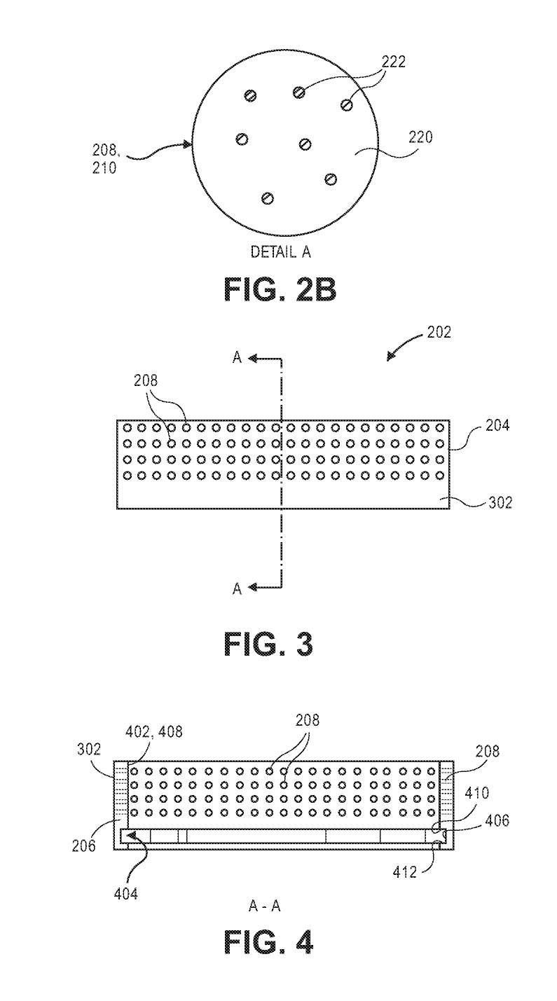

Referring to FIG. 2A, a cross-sectional view of a bottle subassembly including a fragrant component within a cap is shown in accordance with an embodiment. The subassembly can include a fragrant component 202 located within cap 104. In an embodiment, fragrant component 202 includes a sleeve 204. Sleeve 204 may be configured to fit between cap 104 and bottle 102, e.g., within a space between screwtop 114 and finish 108, and thus, sleeve 204 may have a generally cylindrical shape. More particularly, sleeve 204 may have a tubular shape, including an internal passage that is open at an upper end and a lower end. Accordingly, sleeve 204 may have a sleeve wall 206 between an inner diameter and an outer diameter. Sleeve 204 may include a hole 208 in sleeve wall 206. For example, one or more holes 208 may extend from the inner diameter to the outer diameter through sleeve wall 206. In an embodiment, several holes 208 are filled with a fragrant material system 210, and thus, sleeve 204 can contain fragrant material system 210 to release a predetermined smell when exposed to air.

Referring to FIG. 2B, a detail view, taken from Detail A of FIG. 2A, of a fragrant material system is shown in accordance with an embodiment. Fragrant component 202 can include fragrant material system 210 to release a predetermined fragrance when all or a portion of cap 104, e.g., screwtop 114, is removed from bottle 102. Fragrant material system 210 can have predetermined material characteristics. For example, fragrant material system 210 may be a food-grade compound such that fragrant material system 210 may be incidentally ingested by the user without harm. That is, fragrant material system 210 may be edible. Similarly, fragrant material system 210 may be shelf-stable such that the fragrant material system 210 can be safely stored at room temperature within fragrant component 202 and/or between cap 104 and bottle 102. The constituents of fragrant material system 210 may be naturally or artificially derived.

In an embodiment, fragrant material system 210 is non-flowable at room temperature. For example, fragrant material system 210 can have a thick paste-like consistency. Fragrant material system 210 may have a predetermined viscosity within a temperature range of 60-100° F. The predetermined viscosity may prevent fragrant material system 210 from flowing out of holes 208. That is, the viscosity of fragrant material system 210 can allow fragrant material system 210 to be retained within holes 208 or other features of fragrant component 202 during distribution and use.

Fragrant material system 210 can be a mixture, e.g., a solution, a suspension, or a colloid. For example, fragrant material system 210 may include a mixture of a base substance 220 and a fragrant substance 222. Base substance 220 may impart bulk material properties to fragrant material system 210. For example, base substance 220 may be a natural or synthetic wax that is hydrophobic. Accordingly, base substance 220 may make fragrant material system 210 non-water soluble. That is, one or more of the constituents of fragrant material system 210 may not be dissolved by beverage 110.

In an embodiment, fragrant material system 210 is a suspension of fragrant substance 222 within base substance 220. Fragrant substance 222 may include micron-scale or millimeter-scale particles of fragrant materials that have a predetermined food smell. For example, the chemical compounds included in the fragrant substance 222 may smell like a cherry, a lemon, a lime, vanilla, cola, or a berry, such as a blackberry. The fragrant substance 222 may be complementary to the taste of beverage 110 in bottle 102. For example, the following beverage and fragrance pairings may be used: water and citrus or berry fragrance; iced tea and lemon fragrance; lemonade and lime fragrance; root beer soda and vanilla cream fragrance; cola soda and cherry fragrance; lager beer and lime fragrance; wheat beer and lemon fragrance; or margarita alcoholic beverage and salt with lime fragrance.

Fragrant material system 210 may include one or more flavorings. For example, fragrant substance 222 may have a predetermined taste, or a different flavoring substance (not shown, however, a flavoring substance particle may be substituted for one or more of the fragrant substance particles 222 in FIG. 2B) may be mixed with base substance 220 and fragrant substance 222 in fragrant material system 210. For instance, fragrant material system 210 may include a flavoring substance configured to impart a tart acidic taste, such as acidic acid or malic acid. Fragrant material system 210 may include a flavoring substance configured to impart a sweet taste, such as natural cane sugar or the artificial sweetener sucralose. Fragrant material system 210 may include a flavoring substance configured to impart a salty taste, such as table salt or sea salt. The flavoring substance may be complementary to the taste of beverage 110 in bottle 102. For example, the following beverage and flavoring pairings may be used: water and citrus or berry flavor; iced tea and lemon flavor; lemonade and lime flavor; root beer soda and vanilla cream flavor; cola soda and cherry flavor; lager beer and lime flavor; wheat beer and lemon flavor; or margarita alcoholic beverage and salt with lime flavor.

The above examples are provided by way of illustration and not limitation, and it will be understood that fragrant material system 210 may incorporate one or more of a fragrant substance 222 or a flavoring substance having respective predetermined molecular structures. The predetermined molecular structure may cause a user to experience a predetermined smell or taste when drinking from bottle 102. Furthermore, the molecular structures may be different but may evoke a same type of food. For example, fragrant substance 222 may have a first molecular structure that causes the user to experience a predetermined smell to evoke the smell of vanilla cream, and flavoring substance may have a second molecular structure different than the first molecular structure that causes the user to experience a predetermined taste to evoke the taste of vanilla cream. By contrast, the fragrant substance 222 may not evoke the taste of vanilla cream or the flavoring substance may not evoke the smell of vanilla cream.

In addition to mixing base substance 220 with fragrant substance 222 and/or flavoring substance, other additives may be blended into fragrant material system 210. For example, fragrant material system 210 may include color pigments (not shown) to color fragrant material system 210. By way of example, red color pigments may be added to complement a strawberry smell emitted by fragrant substance 222. Fragrant material system 210 may include herbal or medicinal substances (not shown). For example, aloe vera extract may be included in fragrant material system 210 to promote lip health by healing dry or chapped lips. Shea butter may be included in fragrant material system 210 for similar effect. Accordingly, the combinations of substances within fragrant material system 210 as described herein are provided by way of example and not limitation.

Referring to FIG. 3, a side view of a fragrant component having a retention groove is shown in accordance with an embodiment. As described above, fragrant component 202 may include sleeve 204, and sleeve 204 may be tubular. Sleeve 204 may have a sleeve outer surface 302. Sleeve outer surface 302 may be a smooth surface. Accordingly, holes 208 in sleeve wall 206 may be deburred and or formed using a polished compression mold to prevent burrs that could catch on a lip of the user.

Referring to FIG. 4, a cross-sectional view, taken about line A-A of FIG. 3, of a fragrant component having a retention groove is shown in accordance with an embodiment. Hole 208 can be in sleeve wall 206, and may extend entirely through sleeve wall 206. That is, sleeve wall 206 may extend between sleeve outer surface 302 and a sleeve inner surface 402, and hole 208 may be a through-hole extending through sleeve wall 206 from sleeve outer surface 302 to sleeve inner surface 402. Alternatively, hole 208 may not extend entirely through sleeve wall 206. For example, hole 208 may be a blind hole. In an embodiment, hole 208 is a dent, a dimple, or another recess that extends inward from sleeve outer surface 302 toward a sleeve inner surface 402. Sidewalls of holes 208 may be straight, e.g., parallel to an axis of symmetry of hole 208, or the sidewalls may be tapered. Accordingly, a cross-sectional dimension or area of each hole 208 may be constant or variable. Thus, fragrant material system 210 may be loaded into, and retained by, hole 208 in sleeve wall 206.

In an embodiment, sleeve 204 includes a sleeve interlock 404. Sleeve interlock 404 can be a feature of sleeve 204 used to interlock sleeve 204 and bottle 102. More particularly, sleeve interlock 404 can interlock with a mating feature of finish 108 to secure sleeve 204 to bottle 102 when screwtop 114 is removed. By way of example, sleeve interlock 404 may be a groove. The groove can be a retention groove 406 in a mating surface 408 of sleeve 204. Mating surface 408 can be a surface facing an opposing surface of bottle 102. For example, mating surface 408 may be sleeve inner surface 402 facing an outer surface of finish 108. Thus, mating surface 408 may appose, or mate with, a surface of finish 108.

Retention groove 406 of sleeve interlock 404 can receive a mating feature of finish 108. Accordingly, retention groove 406 can include an upper retention flange 410 to limit a downward relative motion between sleeve 204 and finish 108, and a lower retention flange 412 to limit an upward relative motion between sleeve 204 and finish 108. Upper retention flange 410 and lower retention flange 412 may face each other across retention groove 406. Accordingly, retention groove 406 may be a trench formed in sleeve inner surface 402 of sleeve 204. Retention groove 406 can be fabricated around an interior volume of sleeve 204 during a machining or molding, e.g., injection or compression molding, process.

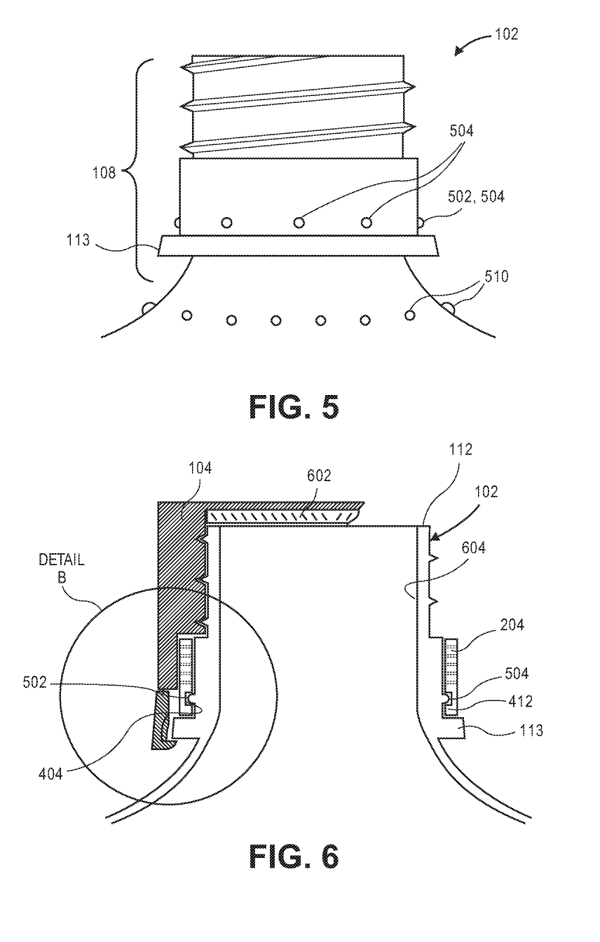

Referring to FIG. 5, a side view of a bottle finish having a retention bump is shown in accordance with an embodiment. Retention groove 406 can mesh or latch with a corresponding feature on finish 108 of bottle 102 to retain sleeve 204 on bottle 102. In an embodiment, bottle 102 includes a finish interlock 502 on finish 108. Finish interlock 502 may include a retention bump 504 to mate with retention groove 406 in mating surface 408 of sleeve interlock 404. Retention bumps 504 may be located around an outer surface of finish 108. For example, retention bumps 504 may be above a locking flange 113 of finish 108. Retention bumps 504 may be asymmetric in shape to allow sleeve 204 to be installed over retention bumps 504 with less mechanical force than is required to remove sleeve 204 from retention bumps 504. For example, retention bumps 504 may taper outward in a downward direction to allow sleeve 204 to slide over a tapered surface until lower retention flange 412 snaps into place below retention bumps 504. Retention bumps 504 can be fabricated using a plastic injection molding process.

In an embodiment, bottle 102 includes one or more witness bumps 510. Witness bump 510 may be molded onto an outer surface of bottle 102, e.g., on a neck or a shoulder region of bottle 102. Witness bumps 510 may be located below locking flange 113. Accordingly, when cap 104 is installed over mouth 112, witness bumps 510 may be visible in an area below cap 104. Witness bumps 510 can be used to verify a quality of bottle assembly 100. For example, a quality assurance inspector may confirm that bottle assembly 100 includes fragrant component 202 based on a presence of witness bumps 510. More particularly, bottles 102 used to manufacture bottle assembly 100 having fragrant component 202 can include witness bumps 510, and thus, when the quality assurance inspector audits a bottling line, the presence of witness bumps 510 can be used to confirm that the bottles 102 being used also include a retention feature, e.g., retention bumps 504, for securing fragrant component 202 to bottle 102.

Referring to FIG. 6, a partial cross-sectional view of a bottle assembly including a fragrant component having a retention groove between a cap and a bottle is shown in accordance with an embodiment. Sleeve 204 can be loaded onto finish 108 by snapping retention groove 406 around retention bumps 504. More particularly, sleeve 204 may be advanced over finish 108 until lower retention flange 412 is between retention bumps 504 and locking flange 113. Locking flange 113 can be a protuberance on a neck of bottle 102. Accordingly, sleeve interlock 404 may be coupled to finish interlock 502 to secure sleeve 204 to finish 108.

Cap 104 may include a flat seal 602. Flat seal 602 may be a liner component fit inside of cap 104 after an initial fabrication of cap 104. The liner component may be formed from a compliant material such that flat seal 602 may act as a gasket to provide a reliable seal against a top edge of mouth 112. That is, flat seal 602 may seal beverage 110 inside of bottle 102 when cap 104 is fastened on finish 108.

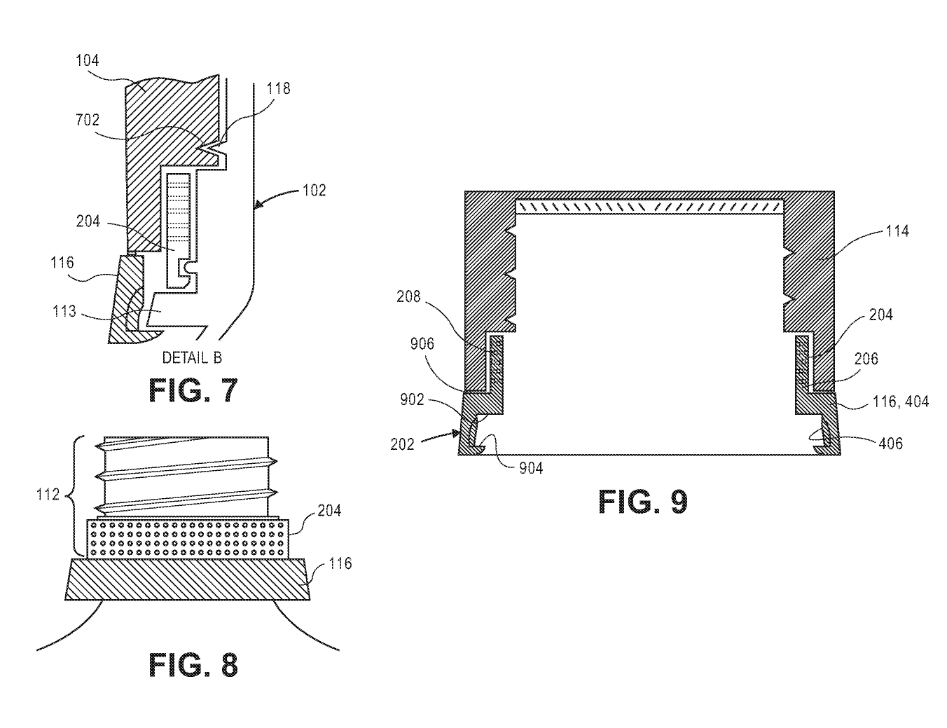

Referring to FIG. 7, a detail view, taken from Detail B of FIG. 6, of a bottle assembly including a fragrant component having a retention groove between a cap and a bottle is shown in accordance with an embodiment. Cap 104 may be installed on bottle 102 over sleeve 204. For example, cap 104 may be screwed into place on bottle 102 until fragrant component 202 is captured between an inner sidewall of cap 104 and an outer surface of mouth 112. Cap 104 can include an internal cap thread 702 to engage with mouth thread 118. Internal cap thread 702 engages with mouth thread 118 such that, when cap 104 is fully installed on mouth 112, a pressure is created between flat seal 602 and a top edge of mouth 112.

When fragrant component 202 includes sleeve 204, sleeve 204 may be enclosed between cap 104 and a portion of finish 108, e.g., mouth 112. Accordingly, fragrant substance 222 loaded within holes 208 in sleeve 204 can be protected between cap 104 and mouth 112 during distribution and prior to use. Fragrant substance 222 of fragrant component 202 can be protected against contact and filth, but may not necessarily be hermetically sealed. In an embodiment, cap 104 includes tamper evident band 116. Tamper evident band 116 can snap into place below locking flange 113 to retain cap 104 on bottle 102 until sufficient removal force is applied by a user.

Referring to FIG. 8, a side view of a tamper evident band and a fragrant component having a retention groove retained on a bottle finish is shown in accordance with an embodiment. The user can unscrew cap 104 to open bottle 102 for drinking. When a consumer opens bottle 102 by removing screwtop 114 from mouth 112, tamper evident band 116 and fragrant component 202 may be retained on finish 108. For example, the line of weakness between tamper evident band 116 and screwtop 114 can break to allow removal of screwtop 114 from mouth 112. Mechanical interference between the flanges around retention groove 406, and retention bumps 504 can limit relative movement between sleeve 204 and finish 108. More particularly, the mechanical interference can retain sleeve 204 on bottle 102. Thus, at least a portion of fragrant component 202, e.g., sleeve 204 and/or fragrant substance 222, can be retained on mouth 112 when screwtop 114 is removed from bottle 102. The aromatic material of fragrant component 202 can stay at bottle 102, and may be exposed, when screwtop 114 is removed. Accordingly, when the consumer takes a drink from a throat of bottle 102, fragrant substance 222 releases a fragrance that can be smelled by the consumer to provide a sensory experience to enhance the drinking experience.

It will be noted that the features of cap 104, fragrant component 202, and bottle 102 described above may be incorporated differently within the components. That is, the features may be swapped between components to perform a similar function as described above. For example, sleeve interlock 404 may include retention bumps, and finish interlock 502 may include a retention groove. It will be appreciated that retention bumps on sleeve inner surface 402 can snap into the retention groove in an outer surface of finish 108, and thus, the configuration described above is provided by way of example and not limitation. The breadth of similar solutions is further illustrated by the embodiments described below

Referring to FIG. 9, a cross-sectional view of a bottle subassembly including a fragrant component integrated with a tamper evident band of a cap is shown in accordance with an embodiment. Sleeve 204 may be integrated with tamper evident band 116, and thus, the monolithic combination may include the features of each individual component. For example, the sleeve portion may include one or more holes 208 in sleeve wall 206, and the tamper evident band 116 portion may include retention groove 406. More particularly, retention groove 406 may be between an upper latching flange 902 and a lower latching flange 904 of the tamper evident band 116 portion. As described below, upper latching flange 902 may function as upper retention flange 410, and lower latching flange 904 may function as lower retention flange 412. Upper latching flange 902 may be a rigid flange, and lower latching flange 904 may be a compliant flange. A compliance of lower latching flange 904 may allow the tamper evident band 116 portion to flex outward when cap 104 is installed over mouth 112. That is, lower latching flange 904 may flex outward to snap onto locking flange 113 of finish 108. After snapping in place, lower latching flange 904 can latch under locking flange 113 to secure fragrant component 202 to bottle 102. A seam 906, i.e., the line of weakness, may join screwtop 114 of cap 104 to fragrant component 202 having the integrated tamper evident band.

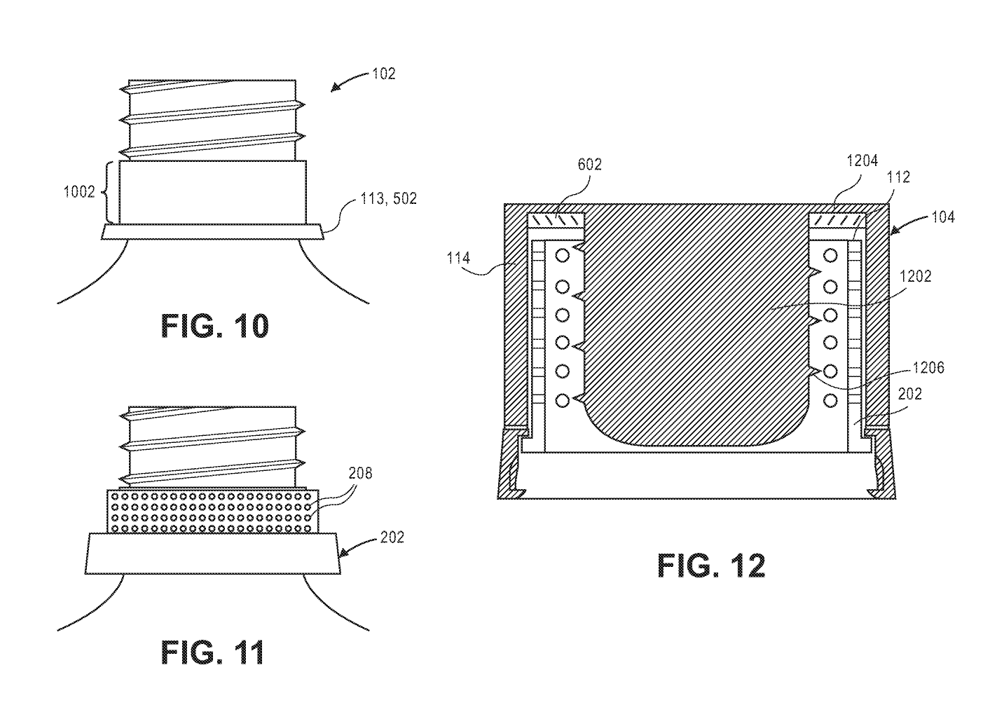

Referring to FIG. 10, a side view of a bottle finish having a locking flange is shown in accordance with an embodiment. Locking flange 113 may be finish interlock 502 of bottle 102. More particularly, in addition to (or instead of) retention bumps 504, bottle 102 may include locking flange 113 to retain fragrant component 202 when screwtop 114 is removed from mouth 112. When cap 104 is installed over finish 108, the integrated tamper evident band 116 may snap over locking flange 113 such that locking flange 113 inserts into retention groove 406 between upper latching flange 902 and lower latching flange 904. Accordingly, locking flange 113 may be between upper retention flange 410 and lower retention flange 412 of fragrant component 202. Similarly, the sleeve portion of fragrant component 202 may be located between screwtop 114 and finish 108 when cap 104 is fastened to bottle 102. For example, sleeve 204 may be between screwtop 114 and an unthreaded sidewall 1002 of mouth 112 when cap 104 is fastened to bottle 102.

Referring to FIG. 11, a side view of a fragrant component integrated with a tamper evident band retained on a bottle finish is shown in accordance with an embodiment. Screwtop 114 can be removed from mouth 112 when a user wants to drink beverage 110 from bottle 102. Upon removal, seam 906 may fracture to separate screwtop 114 from the integrated tamper evident band of fragrant component 202. Accordingly, the sleeve portion may be retained on finish 108 such that fragrant substance 222 within holes 208 can release an aroma. Thus, fragrant component 202 having sleeve 204 integrated with tamper evident band 116 can fulfill a fragrance enhancing function and a tamper alert function.

Referring to FIG. 12, a cross-sectional view of a bottle subassembly including a fragrant component within a cap is shown in accordance with an embodiment. Cap 104 may include a plug 1202 extending downward from a top wall 1204 of screwtop 114. More particularly, plug 1202 may extend along a central axis parallel to sidewalls of screwtop 114. Thus, an annular space may be disposed radially between the side wall of screwtop 114 and plug 1202. Flat seal 602 can include an annular ring shape, e.g., a washer shape, to fit within the annular space. For example, the annular ring may have an inner diameter that conforms to plug 1202 and an outer diameter that conforms to the sidewall of screwtop 114. Thus, the annular ring of flat seal 602 can seal against a top edge of mouth 112 when cap 104 is screwed onto bottle 102.

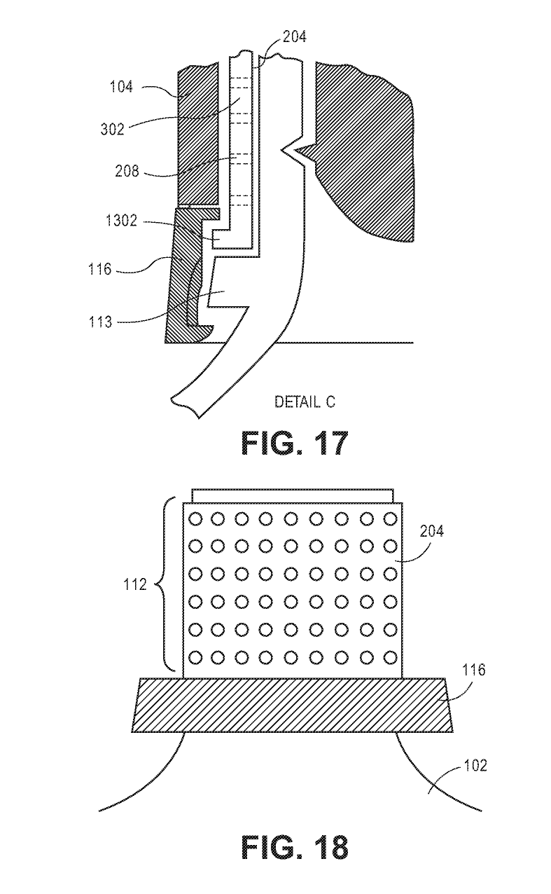

In an embodiment, cap 104 includes a plug thread 1206 extending around an outer surface of plug 1202. Plug thread 1206 may be configured to engage with a throat thread (FIG. 17) of throat 604. Thus, when cap 104 is screwed onto bottle 102, plug 1202 may be located within throat 604 to create an annular space between mouth 112 and the sidewall of screwtop 114. In an embodiment, fragrant component 202 may fit within the annular space.

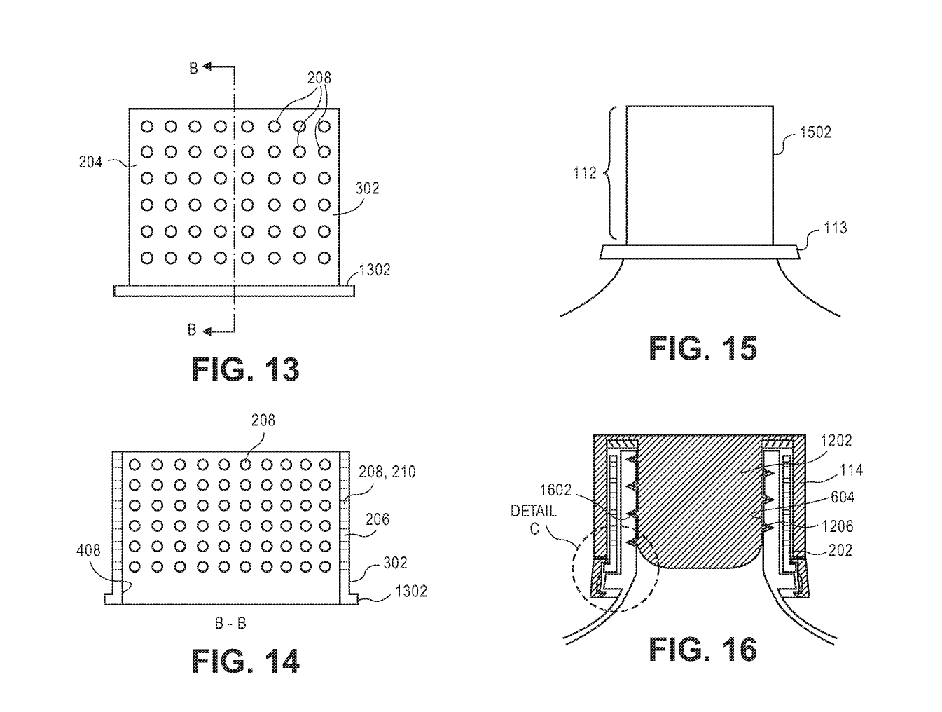

Referring to FIG. 13, a side view of a fragrant component having an outer retention flange is shown in accordance with an embodiment. Fragrant component 202 can include sleeve 204 having holes 208. A retention feature on sleeve 204 may include an outer retention flange 1302. Outer retention flange 1302 can be an annular protuberance extending laterally outward from a bottom end of sleeve 204. Accordingly, outer retention flange 1302 may have an outer diameter greater than an outer diameter of sleeve wall 206.

Referring to FIG. 14, a cross-sectional view, taken about line B-B of FIG. 13, of a fragrant component having an outer retention flange is shown in accordance with an embodiment. The cross-sectional view indicates that holes 208 can extend all or part of the way through sleeve wall 206. Fragrant material system 210 may be loaded into one or more of holes 208, as described above.

Referring to FIG. 15, a side view of a bottle finish is shown in accordance with an embodiment. Mouth 112 of bottle 102 may have a smooth mouth wall 1502. For example, rather than including mouth thread 118, the outer surface of mouth wall 1502 extending between the top edge of bottle 102 and locking flange 113 may include a flat and cylindrical wall.

Referring to FIG. 16, a cross-sectional view of a bottle assembly including a fragrant component between a cap having a plug and a bottle is shown in accordance with an embodiment. In contrast to a smooth mouth wall 1502, finish 108 may include a throat thread 1602 within an inner surface of mouth 112. More particularly, throat thread 1602 may be an internal or external thread formed on a surface of throat 604. Accordingly, finish 108 includes throat 604 within mouth 112, and when plug 1202 of cap 104 is inserted into or located within throat 604, throat thread 1602 engages with a corresponding plug thread 1206 of plug 1202. When cap 104 is fastened to bottle 102, an annular space between mouth wall 1502 and an inner sidewall of screwtop 114 provides a space to hold fragrant component 202. Fragrant component 202 can be protected and enclosed within the space.

Referring to FIG. 17, a detail view, taken from Detail C of FIG. 16, of a bottle assembly including a fragrant component between a cap having a plug and a bottle is shown in accordance with an embodiment. Outer retention flange 1302 of sleeve 204 can be held between tamper evident band 116 of cap 104 and locking flange 113 of finish 108.

Referring to FIG. 18, a side view of a tamper evident band and a fragrant component retained on a bottle finish is shown in accordance with an embodiment. When screwtop 114 is twisted to fracture seam 906 and removed from mouth 112, sleeve 204 may be retained on mouth 112 by tamper evident band 116. It will be appreciated that, when mouth wall 1502 does not require a thread to retain cap 104, sleeve 204 may extend over all or most of a height of mouth wall 1502. That is, fragrant component 202 may have a same height or substantially a same height as mouth 112. Accordingly, a top edge of sleeve 204 may be within 1-10 mm of a top edge of mouth 112. A taller sleeve 204 can allow for a larger volume of fragrant material system 210 to be included between cap 104 and finish 108, and thus, may release more fragrance as compared to a shorter sleeve 204.

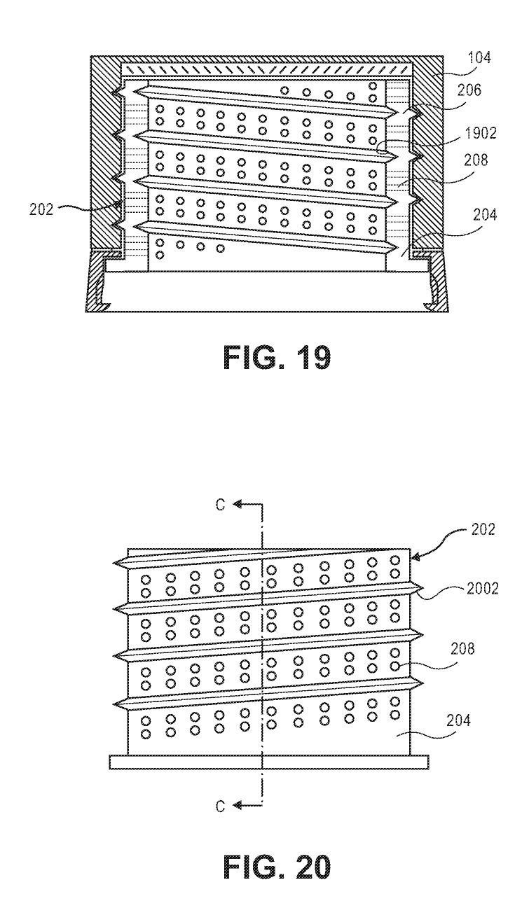

Referring to FIG. 19, a cross-sectional view of a bottle subassembly including a fragrant component screwed into a cap is shown in accordance with an embodiment. Sleeve 204 may be configured to screw onto mouth 112. That is, a retention groove or a retention bump included on sleeve 204 may be a thread 1902. In an embodiment, thread 1902 is an internal thread formed in an inner surface of sleeve wall 206. The internal thread may be referred to as a retention thread 1902 because the internal thread can engage a corresponding thread on mouth 112 to secure sleeve 204 to bottle 102. Thus, sleeve interlock 404 of sleeve 204 can include retention thread 1902 on sleeve wall 206.

Referring to FIG. 20, a side view of a fragrant component having a thread is shown in accordance with an embodiment. In an embodiment, sleeve 204 may include an external thread 2002 to engage with cap 104. More particularly, screwtop 114 of cap 104 may have an internal thread in an inner surface, and the internal thread may screw onto the external thread 2002 of sleeve 204.

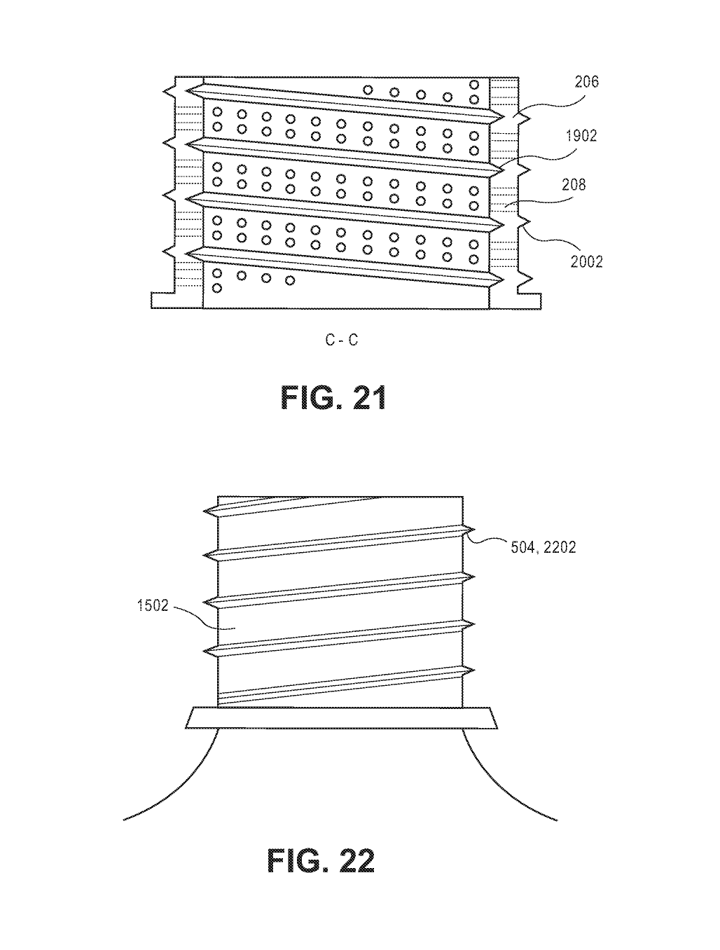

Referring to FIG. 21, a cross-sectional view, taken about line C-C of FIG. 20, of a fragrant component having a thread is shown in accordance with an embodiment. Holes 208 can be formed through sleeve wall 206 between adjacent threads 1902 and adjacent threads 2002. Fragrant material system 210 can be loaded into holes 208 as described above.

Referring to FIG. 22, a side view of a bottle finish having a thread is shown in accordance with an embodiment. Mouth 112 may include a retention feature to mate with retention thread 1902 of sleeve 204. In an embodiment, retention bump 504 of finish interlock 502 is a thread 2202. The retention bump 504 thread can be an external thread extending outward from mouth wall 1502.

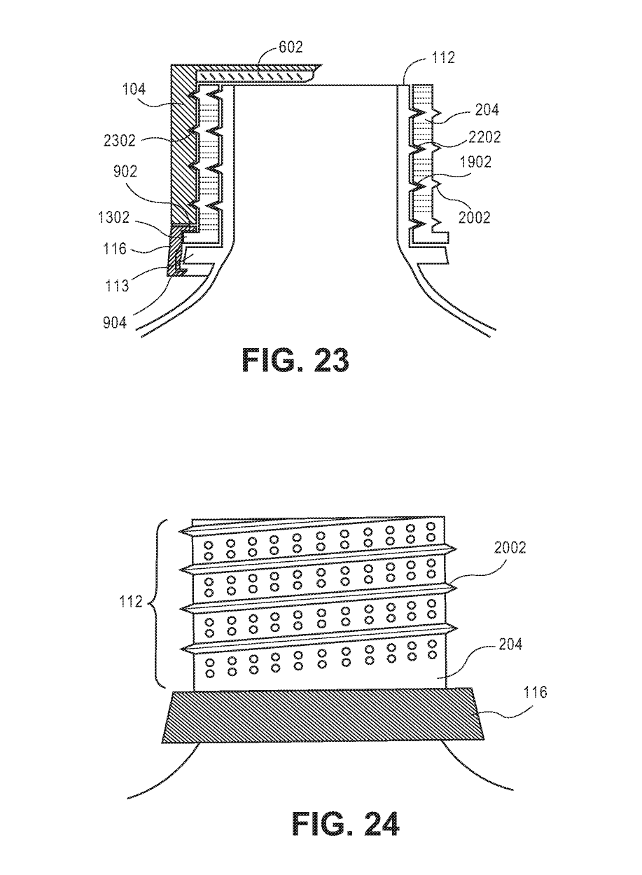

Referring to FIG. 23, a partial cross-sectional view of a bottle assembly including a fragrant component having a thread between a cap and a bottle is shown in accordance with an embodiment. When sleeve 204 is screwed on the bottle 102, thread 2202 of mouth 112 may engage with retention thread 1902 of sleeve 204. Cap 104 can be installed over sleeve 204. More particularly, screwtop 114 can be twisted around sleeve 204 to engage external thread 2002 of sleeve 204 with an internal thread 2302 in screwtop 114. Screwing cap 104 into place can seal flat seal 602 against a top edge of mouth 112, and may snap tamper evident band 116 around locking flange 113 of bottle 102. More particularly, outer retention flange 1302 of sleeve 204 and locking flange 113 of bottle 102 may be captured between upper latching flange 902 and lower latching flange 904 of tamper evident band 116.

Referring to FIG. 24, a side view of tamper evident band and a fragrant component having a thread retained on a bottle finish is shown in accordance with an embodiment. When screwtop 114 is twisted, seam 906 can fracture to separate screwtop 114 from tamper evident band 116. Screwtop 114 can be removed from bottle 102, and tamper evident band 116 may remain in place on locking flange 113 of finish 108. Furthermore, tamper evident band 116 may resist removal of sleeve 204, and sleeve 204 may remain on mouth 112 of bottle 102. Integrating external thread 2002 on sleeve 204 can allow sleeve 204 to have a height that is the same or similar to a height of mouth 112. As described above, maximizing the height of sleeve 204 can allow for more fragrant material system 210 to be loaded onto bottle 102, and thus, may contribute to an enhanced sensory experience when drinking beverage 110 from bottle 102.

In an embodiment, sleeve 204 may be fabricated entirely from fragrant material system 210. For example, base substance 220 of fragrant material system 210 may be a polymer. The polymer can be a food-grade polymer fabricated in any of the shapes described above, and without holes 208. The polymer of base substance 220 can be loaded with fragrant substance 222 to enhance an aroma of the material system. For example, fragrant substance 222 may include particles of a fragrant compound loaded directly into a bulk shape of base substance 220 during a molding process. Accordingly, fragrant component 202 may not include holes 208. Fragrant component 202 can be installed between cap 104 and bottle 102 as described above, and may be retained on bottle 102 when cap 104 or screwtop 114 is removed from mouth 112.

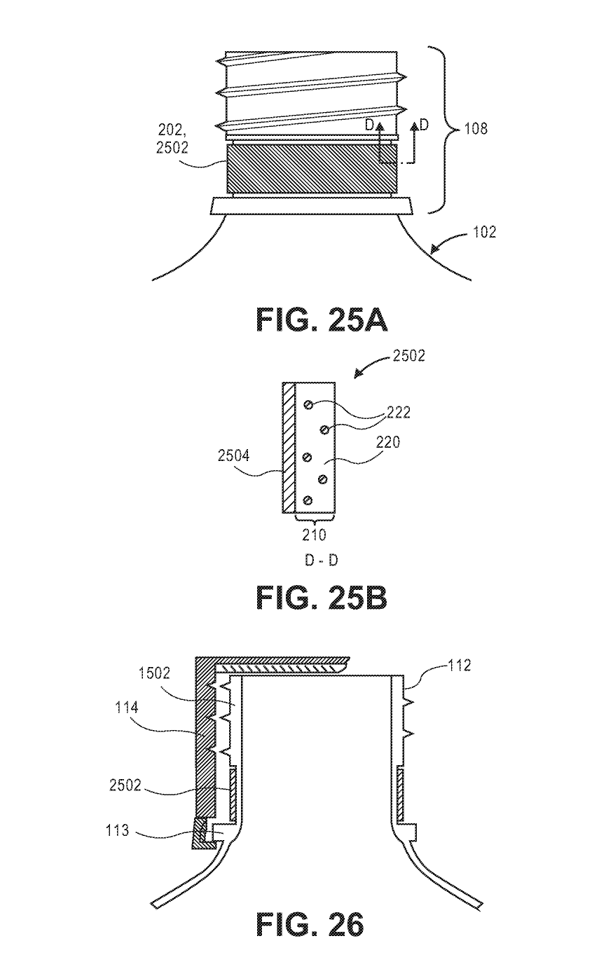

Referring to FIG. 25A, a side view of a fragrant tape on a bottle finish is shown in accordance with an embodiment. Fragrant component 202 may be a fragrant tape 2502. Fragrant tape 2502 can incorporate fragrant material system 210 as described below, and may be applied onto bottle 102. More particularly, fragrant tape 2502 may be adhered to a bottle surface, such as an outer surface of finish 108, to release an aroma when cap 104 is removed from bottle 102.

Referring to FIG. 25B, a cross-sectional view, taken about line D-D of FIG. 25A, of a fragrant tape is shown in accordance with an embodiment. Fragrant tape 2502 may include fragrant material system 210 on an adhesive substrate 2504. For example, adhesive substrate 2504 may be a thin and flexible polymer film having an adhesive substance on at least one side of the film. The polymer film and fragrant tape 2502 may be thin and stretchable. Fragrant material system 210 may be spread, molded, or otherwise loaded onto another side of the film. Accordingly, when fragrant tape 2502 is stuck to finish 108, fragrant material system 210 may face outward from bottle 102. Fragrant material system 210 can include a mixture of fragrant substance 222 and base substance 220, as described above.

In an embodiment, fragrant tape 2502 includes a self-adhesive paper tape. The paper side of the tape may be saturated with a liquid combination of fragrant substance 222 and base substance 220. More particularly, base substance 220 may include a liquid, e.g., an oil, and fragrant substance 222 may be mixed or dissolved in the liquid. Accordingly, fragrant material system 210 may be a liquid mixture or solution trapped within a matrix of material provided by the paper tape, or another porous film material.

Referring to FIG. 26, a partial cross-sectional view of a bottle assembly having a fragrant tape between a cap and a bottle is shown in accordance with an embodiment. Adhesive substrate 2504 may be mounted on mouth 112 by pressing the adhesive side of the film against mouth wall 1502. When fragrant tape 2502 is mounted on bottle 102, fragrant material system 210 may be trapped between screwtop 114 and mouth wall 1502. Corresponding threads of screwtop 114 and mouth 112 may engage to secure cap 104 to bottle 102. Fragrant tape 2502 may be mounted between a lowest thread on mouth 112 and locking flange 113. For example, fragrant tape 2502 may wrap circumferentially around mouth wall 1502. Accordingly, fragrant tape 2502 may be encased and protected between cap 104 and bottle 102.

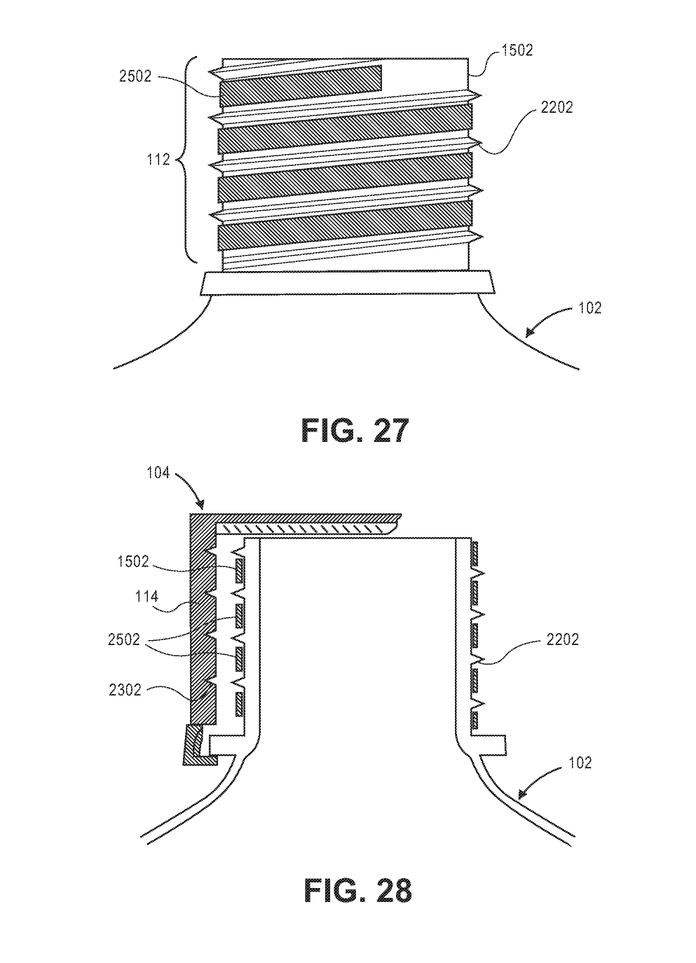

Referring to FIG. 27, a side view of a fragrant tape between threads of a bottle is shown in accordance with an embodiment. Fragrant tape 2502 may wrap spirally around mouth wall 1502. For example, mouth wall 1502 can include an external thread 2202 to engage with cap 104, and fragrant tape 2502 can be wrapped on mouth 112 between adjacent external threads 2202. Accordingly, more fragrant material system 210 may be loaded between cap 104 and bottle 102 as compared to fragrant tape 2502 wrapped circumferentially around mouth 112.

Referring to FIG. 28, a partial cross-sectional view of a bottle assembly having a fragrant tape between a cap and a bottle is shown in accordance with an embodiment. When fragrant tape 2502 is mounted on bottle 102, fragrant material system 210 may be trapped between screwtop 114 and mouth wall 1502. Corresponding threads 2202, 2302 of screwtop 114 and mouth 112 may engage to secure cap 104 to bottle 102. Fragrant tape 2502 mounted between threads 2202 of mouth 112 may be encased and protected between cap 104 and bottle 102.

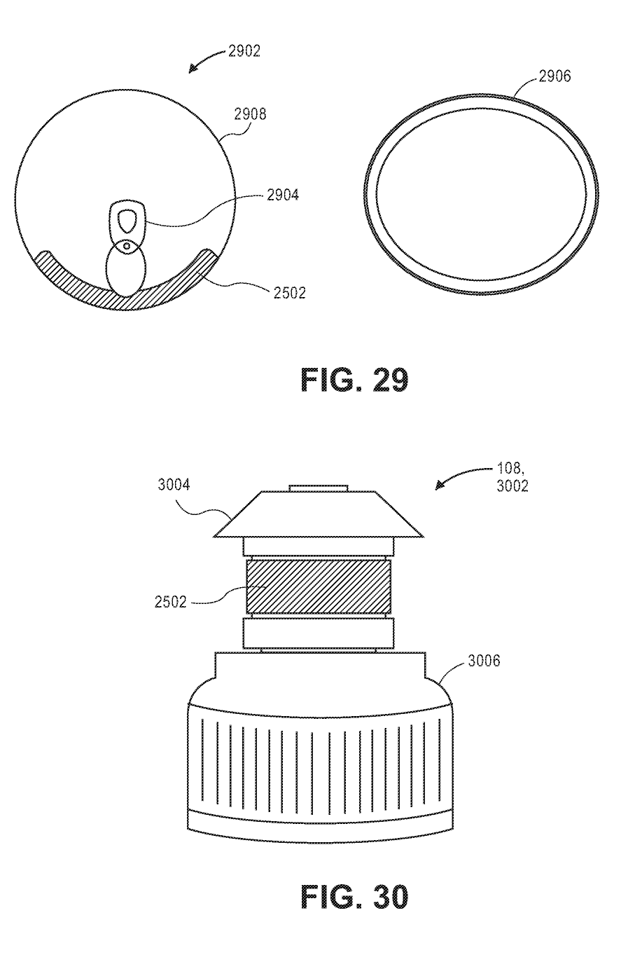

Referring to FIG. 29, a top view of a fragrant tape on a can, and a removed can cover is shown in accordance with an embodiment. The beverage container may be a can 2902. Can 2902 may be an aluminum beverage container, which is known in the art. Can 2902 may be fabricated by automated machines from thin aluminum sheeting. Can 2902 may include a tab 2904, such as a flip tab, used to open a hole in the top of can 2902 for drinking. A flip tab 2904 may remain on can 2902 after opening the beverage container, and thus, may reduce litter as compared to a detachable pull tab type.

The beverage container can include a container cover 2906. Container cover 2906 may, in the case of a can, be a circular piece of material that fits onto can 2902 and protects a top of can 2902 during distribution. For example, container cover 2906 may be a circular sheet of self-adhesive plastic or self-adhesive aluminum foil. Product information may be printed on container cover 2906. Container cover 2906 may be removed from can 2902 prior to opening can 2902 using tab 2904. Container cover 2906 is illustrated in a removed state in FIG. 29.

In an embodiment, fragrant tape 2502 is mounted on can 2902 near the hole through which a user drinks beverage 110 from can 2902. For example, can 2902 may include a can rim 2908 around a top edge of the can, and fragrant tape 2502 may be mounted on can rim 2908. Container cover 2906, when placed on can 2902 over fragrant tape 2502, can protect fragrant material system 210 from abrasion and possible contamination. Removal of container cover 2906 can expose fragrant tape 2502 to release a fragrance when the user drinks from can 2902.

Referring to FIG. 30, a side view of a fragrant tape on a sports cap nipple is shown in accordance with an embodiment. In an embodiment, finish 108 of bottle 102 may be detachable from body 106 of bottle 102. More particularly, finish 108 may include a sports cap 3002. Sports cap 3002 may be fabricated, e.g., by a molding process, separately from body 106. Sports cap 3002 may have a pop-up-to-open design, as is known in the art. Accordingly, sports cap 3002 may have a sports cap nipple 3004 that can fit within, and be moved up and down relative to a threaded base 3006. Threaded base 3006 may include internal threads to attach sports cap 3002 to bottle 102. Tamper evident band 116 may be integrated with threaded base 3006 to allow consumers to validate the integrity of the bottled beverage product.

Fragrant tape 2502 may be mounted on a portion of nipple 3004. The portion may be moved up and down. More particularly, nipple 3004 may be lowered and raised to insert fragrant tape 2502 into threaded base 3006, and to expose fragrant tape 2502 to a surrounding environment. When fragrant tape 2502 is located within threaded base 3006, fragrant material system 210 may be protected from abrasion and contamination. Similarly, when fragrant tape 2502 is exposed, a user may smell fragrant material system 210 when taking a drink from nipple 3004.

In an embodiment, nipple 3004 is fixed relative to threaded base 3006. For example, threaded base 3006 and nipple 3004 may be a monolithic form. In such case, a removable protective cap (not shown) may be mounted on threaded base 3006 around nipple 3004 to protect fragrant tape 2502. When a user wants a drink, the protective cap may be removed to expose fragrant tape 2502, and to allow a drink to be taken from nipple 3004.

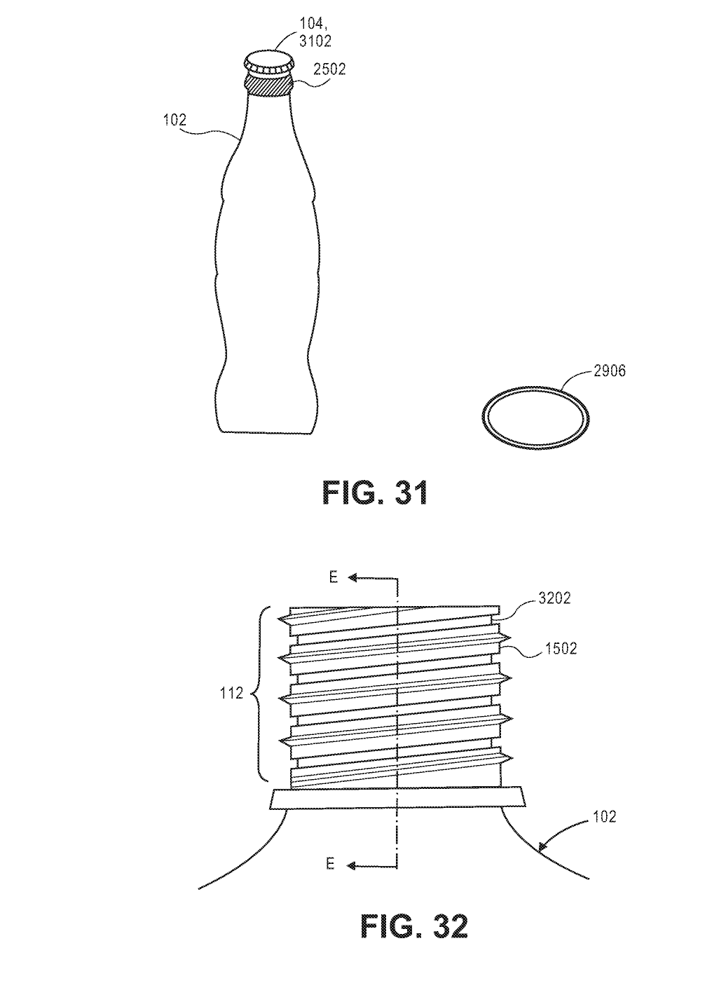

Referring to FIG. 31, a side view of a fragrant tape on a bottle neck, and a removed bottle cover is shown in accordance with an embodiment. The beverage container may be a bottle 102 having a crimp cap 3102. Bottle 102 may be a glass beverage container, which is known in the art. Bottle 102 may be compatible with a metal crimp cap closure. For example, crimp cap 3102 may be a metal pry-off or twist-off crimp cap closure, e.g., a crown style crimp cap closure. A crown style crimp cap closure can be fabricated by a machine stamping a thin disc of metal and inserting a plastic seal gasket inside the crimp cap 3102. Accordingly, crimp cap 3102 closure can seal an opening of bottle 102, and may be designed for permanent or replaceable removal from bottle 102. Bottle 102 having crimp cap 3102 may be used for the distribution and sale of a wide variety of beverages including beer, soda, or iced tea. Similarly, all beverage containers described herein may be used for the distribution and sale of a wide variety of beverages including beer, soda, or iced tea.

The beverage container may include a container cover 2906. Container cover 2906 may, in the case of a glass bottle 102, be a circular piece of material that fits onto crimp cap 3102 and protects crimp cap 3102 and fragrant tape 2502 during distribution. For example, container cover 2906 may be a circular sheet of self-adhesive plastic or self-adhesive aluminum foil. Product information may be printed on container cover 2906. Container cover 2906 may be removed from crimp cap 3102 prior to opening bottle 102. Container cover 2906 is illustrated in a removed state in FIG. 31.

In an embodiment, fragrant tape 2502 is mounted on bottle 102 near the hole through which a user drinks beverage 110 from bottle 102. For example, bottle 102 can include a bottle lip below crimp cap 3102, and fragrant tape 2502 may be mounted around finish 108 of bottle 102 below the lip and crimp cap 3102. Container cover 2906, when placed on crimp cap 3102 and over fragrant tape 2502, can protect fragrant material system 210 from abrasion and possible contamination. Removal of container cover 2906 can expose fragrant tape 2502 to release a fragrance when the user drinks from bottle 102.

Referring to FIG. 32, a side view of a bottle finish having a groove is shown in accordance with an embodiment. A groove 3202 can be formed in mouth wall 1502 of bottle 102. For example, groove 3202 may extend around mouth wall 1502 in a spiral between adjacent external threads 2202 of mouth 112.

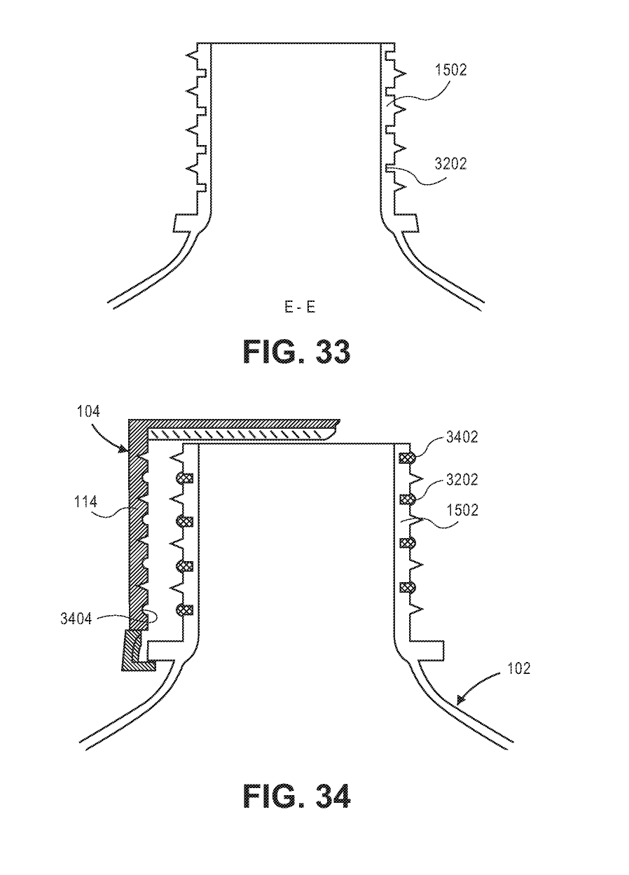

Referring to FIG. 33, a cross-sectional view, taken about line E-E of FIG. 32, of a bottle finish having a groove is shown in accordance with an embodiment. Groove 3202 can include an angular cross-sectional profile. For example, groove 3202 may be a square slot machined or molded into mouth wall 1502. Alternatively, groove 3202 may have a curvilinear profile. For example, the profile of groove 3202 may be semicircular.

Referring to FIG. 34, a partial cross-sectional view of a bottle assembly having a fragrant thread between a cap and a groove of a bottle finish is shown in accordance with an embodiment. Groove 3202 may be partially or completely filled by a fragrant thread 3402. Fragrant thread 3402 can be an elongated wire-like shape formed from fragrant material system 210. For example, fragrant material system 210 may include fragrant substance 222 suspended in a waxy base substance 220, and fragrant material system 210 may be extruded to form fragrant thread 3402. Fragrant thread 3402 may be cut to length and wrapped around mouth wall 1502 inside of groove 3202. Accordingly, when cap 104 is installed on bottle 102, fragrant thread 3402 may be protected between screwtop 114 and mouth wall 1502.

In an embodiment, cap 104 includes a clearance groove 3404 formed in a wall of screwtop 114. More particularly, clearance groove 3404 can be machined or molded into an inner surface of screwtop 114 to provide a lateral clearance for fragrant thread 3402. Clearance groove 3404 may be spiral, and may conform to a path or shape of groove 3202 in mouth wall 1502. When cap 104 is screwed into place, clearance groove 3404 and groove 3202 may be aligned with each other. Thus, fragrant thread 3402 may remain separated from screwtop 114 during placement and removal of cap 104 on bottle 102, and a likelihood that fragrant thread 3402 will be dislodged or disturbed during installation of cap 104 may be reduced.

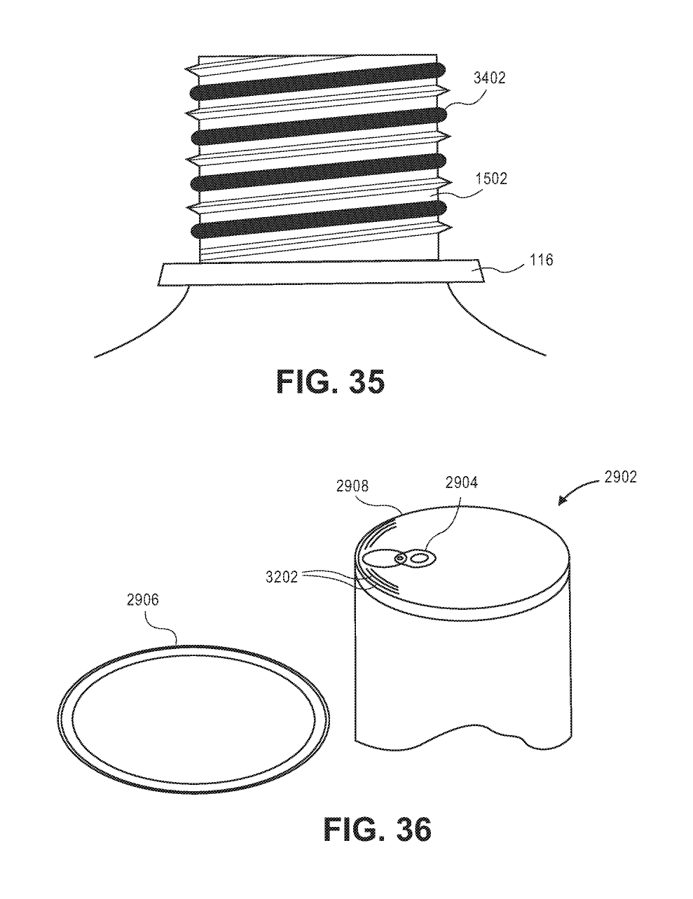

Referring to FIG. 35, a side view of a fragrant thread retained in a groove of a bottle finish is shown in accordance with an embodiment. When screwtop 114 is removed from mouth 112 of bottle 102, fragrant thread 3402 may be retained within groove 3202. Fragrant thread 3402 may be exposed to the senses of a user, and the user may smell fragrant thread 3402 when drinking from bottle 102. As shown, fragrant thread 3402 may remain well-formed and may not be smeared by screwtop 114 during removal from bottle 102. More particularly, clearance groove 3404 may pass over fragrant thread 3402 during removal to avoid scraping or spreading fragrant thread 3402 over mouth wall 1502.

Referring to FIG. 36, a perspective view of a fragrant material system retained in a groove on a can, and a removed can cover is shown in accordance with an embodiment. Groove 3202 may be formed in a top of can 2902. One or more grooves 3202 can be formed in the top by grinding, stamping, or otherwise deforming the top to create grooves 3202. Grooves 3202 may be near can rim 2908, and may be parallel to can rim 2908. For example, when can rim 2908 is circular, grooves 3202 may be semi-circular. Grooves 3202 may be nested, i.e., several grooves 3202 may be parallel to each other over a groove path between opposite ends of each groove. In an embodiment, fragrant material system 210 can be loaded into grooves 3202 on can 2902. For example, fragrant thread 3402 may be inserted into grooves 3202. Fragrant material system 210 may be pressed into grooves 3202 such that the grooved recesses retain fragrant material system 210 when the user drinks from can 2902. A container cover 2906 may be placed over can 2902 to protect fragrant material system 210 during distribution, and may be removed from can 2902 to expose tab 2904 and fragrant material system 210.

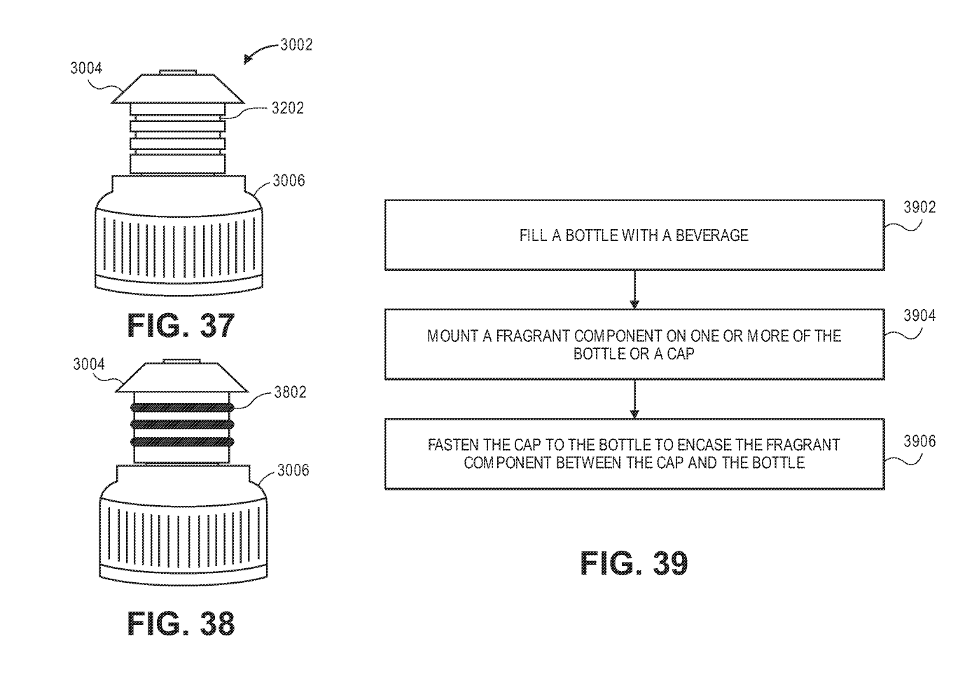

Referring to FIG. 37, a side view of a sports cap nipple having a groove is shown in accordance with an embodiment. Sports cap 3002 can include groove 3202 to retain fragrant materials system. In an embodiment, several grooves 3202 extend circumferentially around nipple 3004 of sports cap 3002. Each groove 3202 can be machined or molded into nipple 3004. When nipple 3004 is moved downward relative to threaded base 3006, grooves 3202 may be hidden within an encased by threaded base 3006. Similarly, when nipple 3004 is moved upward relative to threaded base 3006, grooves 3202 may be exposed to the surrounding environment.

Referring to FIG. 38, a side view of a fragrant ring retained in a groove of a sports cap nipple is shown in accordance with an embodiment. One or more fragrant rings 3802 can be inserted into respective grooves 3202 of nipple 3004. Fragrant ring 3802 may be similar in construction to fragrant thread 3402, however, fragrant ring 3802 may have an annular shape. Fragrant ring 3802 may be elastic such that it can be widened to be placed around nipple 3004 and released to contract to a smaller diameter and insert into groove 3202. Similarly, fragrant ring 3802 may be a fragrant thread 3402 having two ends and a length that is equal to a circumference of groove 3202. Accordingly, fragrant ring 3802 can be wrapped around nipple 3004 to fill groove 3202. Fragrant rings 3802 can be hidden and protected within threaded base 3006 when nipple 3004 is pressed downward, or fragrant rings 3802 can be exposed to the surrounding environment when nipple 3004 is pulled upward.

Referring to FIG. 39, a flowchart of a method of manufacturing a bottle assembly having a fragrant component is shown in accordance with an embodiment. At operation 3902, bottle 102 is filled with beverage 110. Beverage 110 can be any liquid for human consumption, e.g., non-alcoholic or alcoholic drink. At operation 3904, fragrant component 202 is mounted on one or more of bottle 102 or cap 104. Fragrant component may contain fragrant material system 210. For example, fragrant component 202 may include sleeve 204 containing fragrant material system 210 in holes 208, and sleeve 204 may be snapped onto retention bumps 504 of finish 108 (FIG. 6). Alternatively, fragrant component 202 may be a part of cap 104 or snapped into place on cap 104. For example, fragrant component 202 may be sleeve 204 integrated with tamper evident band 116 of cap 104 (FIG. 9). At operation 3906, cap 104 is fastened to bottle 102 to encase fragrant component 202 between cap 104 and bottle 102. Cap 104 may also seal beverage 110 within bottle 102 to allow the packaged beverage 110 to be delivered to a customer or a consumer.

Fragrant material system 210 may be integrated into bottle assembly 100 in various manners. For example, in several of the embodiments described above, fragrant material system 210 is located in a hole or a groove. Accordingly, the method of manufacturing bottle assembly 100 can include loading fragrant material system 210 into a receiving recess, e.g., into hole 208 in sleeve 204. Loading fragrant material system 210 into the receiving recess can be achieved using various techniques. For example, sleeve 204 having holes 208 may be dipped into a paste containing fragrant material system 210 such that the paste fills holes 208 to load the holes 208 with fragrant material system 210. The structures and methods described above, however, are illustrative and not limiting. Other manners of encasing fragrant material system 210 between cap 104 and bottle 102 are described below. In particular, fragrant material system 210 can be incorporated onto bottle 102 using various indirect or direct methodologies.

An example of an indirect methodology of applying fragrant material system 210 to bottle 102 includes incorporating fragrant material system 210 in cap 104 of a bottle assembly 100. For example, fragrant material system 210 may be loaded into sleeve 204 and then sleeve 204 may be incorporated into cap 104 prior to bottling (FIG. 12). During bottling, an automated bottling line can handle cap 104 containing fragrant material system 210 in much the same way that the line would handle a typical plastic screw cap 104. For example, after filling bottle 102 with beverage 110, an automated capping machine may perform an operation of screwing cap 104 containing fragrant material system 210 onto finish 108. Tamper evident band 116 of cap 104 can snap onto locking flange 113 to secure fragrant material system 210 onto bottle 102.

Another indirect methodology of applying fragrant material system 210 to bottle 102 includes incorporating a volume of fragrant material system 210 onto a surface inside of cap 104. For example, fragrant material system 210 including base substance 220 and fragrant substance 222 may be spread onto a threaded surface within cap 104. When cap 104 is installed onto bottle 102, a portion of fragrant material system 210 may be forced into contact with a threaded surface of finish 108. Accordingly, when screwtop 114 is unscrewed and removed from finish 108, the portion of fragrant material system 210 may adhere to finish 108 and remain on bottle 102. The operation of applying fragrant material system 210 to the inside surface of cap 104 can be integrated into a process flow of fabricating cap 104. For example, after a compression molding process, a folding process, or a slitting process, fragrant material system 210 may be applied to the inside surface of cap 104. Alternatively, fragrant material system 210 may be applied to the inside surface of cap 104 in a separate automated process after fabrication of cap 104 is complete.

Indirect methodologies of applying fragrant material system 210 to bottle 102 can also include selling pre-flavored screwtop 114 to consumers prior to installation on bottle 102. For example, fragrant material system 210 can be incorporated onto screwtop 114, and the loaded screwtops 114 can be marketed and sold as stand-alone beverage enhancing products. A consumer may purchase a bottled beverage of their choice, which is sealed by a typical bottle cap. The consumer may remove the typical cap and replace the typical cap with a screwtop 114 containing fragrant material system 210. When the new screwtop 114 is installed, fragrant material system 210 may be installed onto finish 108. Accordingly, when the new screwtop 114 is removed, e.g., by twisting screwtop away from finish 108, a portion of fragrant material system 210 may remain on bottle 102 and the consumer may detect the predetermined smell of fragrant material system 210 when taking a drink from bottle 102.

A direct methodology of applying fragrant material system 210 to bottle 102 can include applying fragrant material system 210 to the threads on finish 108 before installing a cap 104. For example, fragrant material system 210 may be packaged in a pouch, tube, or other packaging used to dispense a liquid or pasty substance. The packaging can be opened by a user, and fragrant material system 210 may be dispensed onto bottle 102. For example, fragrant material system 210 may be squeezed out of the packaging and spread onto the threads on finish 108. Fragrant material system 210 can be applied in a volume that is detectable by the user when taking a drink from bottle 102. Dispensing fragrant material system 210 as a liquid or pasty substance onto bottle 102 can be performed as an operation of an automated bottling line before or after filling bottle 102 with beverage 110. Furthermore, spreading fragrant material system 210 on bottle 102 can be performed prior to capping bottle 102, e.g., at a beginning of an automated bottling line.

Another direct methodology of applying fragrant material system 210 to bottle 102 can include dispensing fragrant material system 210 from a package by a consumer. For example, a pouch or tube of a liquid or pasty fragrant material system 210 can be opened by a consumer and dispensed onto bottle 102. The consumer can purchase a bottled beverage having a typical cap, and the cap can be removed to expose finish 108 to the consumer. The consumer may purchase fragrant material system 210 packaged in a squeeze tube or similar packaging. The consumer can then open the small tube and spread the enclosed fragrant material system 210 on mouth 112.

In any of the embodiments described above, when a consumer opens bottle assembly 100 to drink beverage 110, and raises mouth 112 to the consumer's lips to drink, the consumer may observe an appealing fragrant aroma emitted by fragrant material system 210. The aroma can enhance the drinking experience by creating a synergy between a taste of beverage 110 and a smell of fragrant material system 210. When beverage 110 is weakly flavored or non-flavored, e.g., when beverage 110 is water, the aroma can create a more stimulating drinking experience. Thus, the consumer may enjoy drinking from bottle assembly 100 more than drinking from a typical, unenhanced bottle. If the consumer decides not to finish beverage, consumer may reinstall screwtop 114 onto bottle 102 to cover and protect fragrant material system 210 until a next drink is desired.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

1. A bottle assembly, comprising:
a bottle including a finish having a mouth;
a cap mounted on the bottle over the mouth; and
a fragrant component between the cap and the finish, wherein the fragrant component includes a sleeve and a fragrant material system in the sleeve, wherein the sleeve is tubular, and wherein the fragrant material system is non-flowable at room temperature.

2. The bottle assembly of claim 1, wherein the sleeve includes a tubular wall enclosed between the cap and the finish, and wherein the tubular wall contains the fragrant material system.

3. The bottle assembly of claim 2, wherein the sleeve includes a hole in the tubular wall, and wherein the fragrant material system is in the hole.

4. The bottle assembly of claim 2, wherein the sleeve includes a sleeve interlock coupled to a finish interlock of the finish.

5. The bottle assembly of claim 4, wherein one or more of the sleeve interlock or the finish interlock includes a retention groove in a mating surface.

6. The bottle assembly of claim 5, wherein one or more of the sleeve interlock or the finish interlock includes a retention bump to mate with the retention groove in the mating surface.

7. The bottle assembly of claim 6, wherein the retention bump is a thread.

8. The bottle assembly of claim 5, wherein the sleeve interlock includes the retention groove between an upper retention flange and a lower retention flange, and wherein the finish interlock includes a locking flange in the retention groove between the upper retention flange and the lower retention flange.

9. (canceled)

10. The bottle assembly of claim 1, wherein the finish includes a throat within the mouth, wherein the cap includes a plug within the throat, and wherein the finish includes a throat thread engaged with a plug thread of the cap.

11. The bottle assembly of claim 1, wherein at least a portion of the fragrant component is retained on the finish when the cap is removed from the bottle.

12. A fragrant component, comprising:
a sleeve having
a sleeve wall between a sleeve outer surface and a sleeve inner surface, wherein
the sleeve wall is tubular,
a hole in the sleeve wall, and
a sleeve interlock; and
a fragrant material system in the hole, wherein the fragrant material system is non-flowable at room temperature.

13. The fragrant component of claim 12, wherein the sleeve interlock includes a retention groove in the sleeve wall.

14. The fragrant component of claim 12, wherein the sleeve interlock includes a retention thread on the sleeve wall.

15. The fragrant component of claim 12, wherein the sleeve interlock includes a tamper evident band having an upper latching flange and a lower latching flange.

16. The fragrant component of claim 12, wherein the fragrant material system is non-water soluble.

17. The fragrant component of claim 16, wherein the fragrant material system includes a mixture of a base substance and a fragrant substance, wherein the mixture is edible, and wherein the fragrant substance has a food smell.

18. A bottle assembly, comprising:
a bottle including a finish having a mouth;
a cap mounted on the bottle over the mouth; and
a sleeve between the cap and the finish, wherein the sleeve is tubular, wherein the sleeve contains a fragrant material system, and wherein the fragrant material system is non-flowable at room temperature.

19. The bottle assembly of claim 18, wherein the sleeve includes a hole in a tubular wall, and wherein the fragrant material system is in the hole.

20. The bottle assembly of claim 18, wherein the sleeve includes a sleeve interlock coupled to a finish interlock of the finish.

21. The bottle assembly of claim 20, wherein one or more of the sleeve interlock or the finish interlock includes a retention groove in a mating surface.

22. The bottle assembly of claim 21, wherein one or more of the sleeve interlock or the finish interlock includes a retention bump to mate with the retention groove in the mating surface.

23. The bottle assembly of claim 22, wherein the retention bump and the retention groove are mating threads.

24. The bottle assembly of claim 21, wherein the sleeve interlock includes the retention groove between an upper retention flange and a lower retention flange, and wherein the finish interlock includes a locking flange in the retention groove between the upper retention flange and the lower retention flange.

25. The bottle assembly of claim 18, wherein the finish includes a throat within the mouth, wherein the cap includes a plug within the throat, and wherein the finish includes a throat thread engaged with a plug thread of the cap.

26. The bottle assembly of claim 18 further comprising a tamper evident band around the finish, wherein the tamper evident band includes an upper latching flange and a lower latching flange, and wherein the sleeve includes an outer retention flange between the upper latching flange and the lower latching flange.

27. A method, comprising:
mounting a fragrant component on one or more of a bottle or a cap, wherein the fragrant component includes a sleeve and a fragrant material system in the sleeve, wherein the sleeve is tubular, and wherein the fragrant material system is non-flowable at room temperature; and
fastening the cap to the bottle to encase the fragrant component between the cap and the bottle.

28. The method of claim 27, wherein mounting the fragrant component includes mounting the sleeve containing the fragrant material system on a finish of the bottle.

29. The method of claim 28 further comprising loading the fragrant material system into a hole in a tubular wall of the sleeve.

30. The method of claim 27 further comprising filling the bottle with a beverage.
